(19) United States
(12) Patent Application Publication
(10) Pub. No.: US 2011/0152622 A1
(43) Pub. Date: Jun. 23, 2011

(54) SURGICAL ACCESS APPARATUS WITH CONSTRAINING MECHANISM

(76) Inventors: Oivind Brockmeier, Medford, MA (US); Jared Alden Judson, Medford, MA (US); Kenneth Allen Focht, Needham, MA (US)

(21) Appl. No.: 12/961,526

(22) Filed: Dec. 7, 2010

Related U.S. Application Data
(60) Provisional application No. 61/287,837, filed on Dec. 18, 2009.

(51) Int. Cl. A61B 1/32

(52) U.S. Cl. 600/201

(57) ABSTRACT

A surgical access apparatus includes a housing member, a portal member extending from the housing member and defining a central longitudinal axis, and defining a longitudinal passage therethrough dimensioned to permit passage of an surgical object and a constraining mechanism associated with at least one of the housing member and the portal member. The constraining mechanism includes a constraining member dimensioned and positioned to intersect the longitudinal passage. The constraining member is adapted to engage the surgical object and bias the surgical object toward a position radially displaced relative to the central longitudinal axis. The constraining member is adapted to transition from an initial condition in the absence of the surgical object to a stressed condition in the presence of the surgical object. The constraining member may be normally biased toward the initial condition.

### CROSS REFERENCE TO RELATED APPLICATION

The present application claims the benefit of and priority to U.S. Provisional Application Ser. No. 61/287,837 filed on Dec. 18, 2009, the entire contents of which are incorporated herein by reference.

### BACKGROUND

1. Technical Field

The present disclosure relates to a surgical portal for accessing underlying body tissue to permit the introduction of surgical objects in conjunction with a medical procedure. More particularly, the present disclosure relates to a surgical portal including a constraining mechanism for effecting the positioning and/or stabilizing of a surgical object with respect to the surgical portal.

2. Discussion of Related Art

Surgical portals are employed in various minimally invasive procedures including laparoscopic or endoscopic procedures. Such portals are inclusive of trocar cannulas, catheters, or, in the event of a minimally invasive hand assist procedures, hand access devices. These portals typically incorporate a seal to form a fluid tight seal about an instrument or hand passed through the portal. These seals are often limited by their ability to sustain a seal when an instrument, particularly, a smaller diameter instrument, is moved off-axis relative to a central axis of the portal.

During a typical surgical procedure, several portals may be positioned to access an underlying body cavity. The clinician may move from portal to portal to perform the desired surgical task(s). One disadvantage of this approach is that an instrument within a portal may be unattended and subject to dislodgement, particularly, when accessing a pressurized body cavity.

### SUMMARY

Accordingly, a surgical access apparatus having the ability to constrain movement of a surgical instrument to minimize dislodgement and potentially improve the sealing capacity is provided. In accordance with one embodiment, the surgical access apparatus includes a housing member, a portal member extending from the housing member and defining a longitudinal passage therethrough dimensioned to permit passage of a surgical object and a constraining mechanism associated with at least one of the housing member and the portal member. The constraining mechanism includes a constraining member dimensioned and positioned to intersect the longitudinal passage. The constraining member is adapted to engage the surgical object and potentially retain the surgical object in fixed relation with respect to the central longitudinal axis. The constraining member may be adapted to bias the surgical object toward a position radially displaced relative to the central longitudinal axis. The constraining member is adapted to transition from an initial condition in the absence of the surgical object to a stressed condition in the presence of the surgical object, and may be normally biased toward the initial condition.

In one embodiment, the constraining member includes an elongated spring member. The elongated spring member may be at least partially disposed within the portal member. The elongated spring member includes leading and trailing ends. One of the leading and trailing ends may be mounted in fixed relation to the portal member. The other of the leading and trailing end may be mounted for longitudinal movement relative to the portal member. The elongated spring member may define a bowed segment between the leading and trailing ends, and may be dimensioned to engage and substantially restrain the surgical object against an inner wall surface of the portal member.

In another embodiment, the constraining member defines a substantially disc shaped member. The disc shaped member has an opening therethrough for substantially restraining the surgical object. The opening may be radially displaced with respect to the central longitudinal axis of the portal member. The disc shaped member further has inner surfaces defining an object passage in communication with the opening. The inner surfaces may be dimensioned and configured to bias the surgical object toward the opening. The inner surfaces may be arranged to taper inwardly toward the opening.

In another embodiment, the constraining member includes a lever mounted within one of the housing and the portal member. The lever may be mounted for pivotal movement between a first position corresponding to the initial condition of the constraining member and a second position corresponding to the stressed condition of the constraining member. The lever may be normally biased toward the first position. An eyelet may be associated with the constraining member. The eyelet may define an opening for passage of the surgical object. The lever may be dimensioned to engage and substantially restrain the surgical object against an inner wall surface of the eyelet. The lever may be dimensioned to engage the eyelet, and cooperate with the eyelet to substantially close the longitudinal passage of the portal member when in the first position thereof.

An object seal may be disposed within the housing. The object seal may be dimensioned to establish a substantial seal about the surgical object. A zero closure valve may be adapted to open to permit passage of the surgical object and to close in the absence of the surgical object.

### BRIEF DESCRIPTION OF THE DRAWINGS

Preferred embodiments of the present disclosure will be better appreciated by reference to the drawings wherein:

FIG. 1 is a perspective view of the surgical access apparatus in accordance with the principles of the present disclosure;

FIG. 1A is a side cross-sectional view of the surgical access apparatus taken along the lines 1A-1A of FIG. 1;

FIG. 2 is a side cross-sectional view of the surgical access apparatus taken along the lines 2-2 of FIG. 1 illustrating the constraining mechanism in the form of an elongated spring and depicted in an initial condition in the absence of a surgical object;

FIG. 3 is a top plan view of the elongated spring illustrating the mounting mechanism for mounting the elongated spring to the internal wall of the portal member;

FIG. 4 is a perspective view of the elongated spring;

FIG. 5 is a view similar to the view of FIG. 4 illustrating the elongated spring in a stressed condition in the presence of a surgical object.

FIG. 6 similar to the view of FIG. 3 illustrating the arrangement of the mounting mechanism when the elongated spring is in the stressed condition;

FIG. 7 is an axial view taken along the lines 7-7 of FIG. 5 illustrating the elongated spring constraining the surgical object;

FIG. 8 is an axial view illustrating the elongated spring constraining an instrument having a relatively small diameter;

FIG. 9 is a cross-sectional view of an alternative embodiment of the present disclosure illustrating the constraining mechanism in the form of a constraining disc;

FIG. 10 is a view similar to the view of FIG. 9 illustrating introduction of a surgical object within the constraining disc of FIG. 9;

FIG. 11 is a view similar to the view of FIG. 10 illustrating the constraining disc biasing the surgical object in a radial outward direction to be constrained within the radial displaced opening;

FIG. 12 is a side cross sectional view of a segment of the portal member illustrating another alternate embodiment of the constraining mechanism in the form of a lever and associated constraining eyelet with the lever in a first position;

FIG. 13 is a cross-sectional view taken along the lines 13-13 of FIG. 12 illustrating the lever mounted within the constraining eyelet;

FIG. 14 is a perspective view of the eyelet of the constraining mechanism of FIGS. 12 and 13;

FIG. 15 is a partial cross-sectional view illustrating a surgical object passing through the constraining mechanism with the lever in a second pivoted position and biasing the surgical object radially outwardly against the constraining eyelet; and

FIG. 16 is a perspective view further illustrating the surgical object secured the within the constraining mechanism.

### DETAILED DESCRIPTION OF THE EMBODIMENTS

The access apparatus of the present disclosure is capable of accommodating objects of varying diameters, e.g., including instruments from about 2.0 millimeter (mm) to about 15 millimeter (mm), during a minimally invasive surgical procedure. Moreover, the access apparatus 100 contemplates the introduction and manipulation of various types of instrumentation adapted for insertion through a trocar and/or cannula assembly while maintaining a fluid tight interface about the instrumentation to prevent gas and/or fluid leakage from the established pneumoperitoneum so as to preserve the atmospheric integrity of a surgical procedure. Specifically, the access apparatus 10 includes a constraining mechanism for selectively retraining the surgical object in a predetermined fixed position with respect to the apparatus. This feature of the present disclosure desirably minimizes the potential of inadvertent movement or dislodgement of the surgical object, and may assist in maintaining a seal within the apparatus.

Examples of surgical objects or instrumentation contemplated for use with the access apparatus 100 include clip appliers, graspers, dissectors, retractors, staplers, laser probes, photographic devices, endoscopes and laparoscopes, tubes, and the like. Such instruments will be collectively referred to herein as “surgical objects”.

In the following discussion, the term “proximal” or “trailing” will refer to the portion of the access apparatus nearest to the clinician during the surgical procedure while the term “distal” or “leading” will refer to that portion of the access apparatus most remote to the clinician.

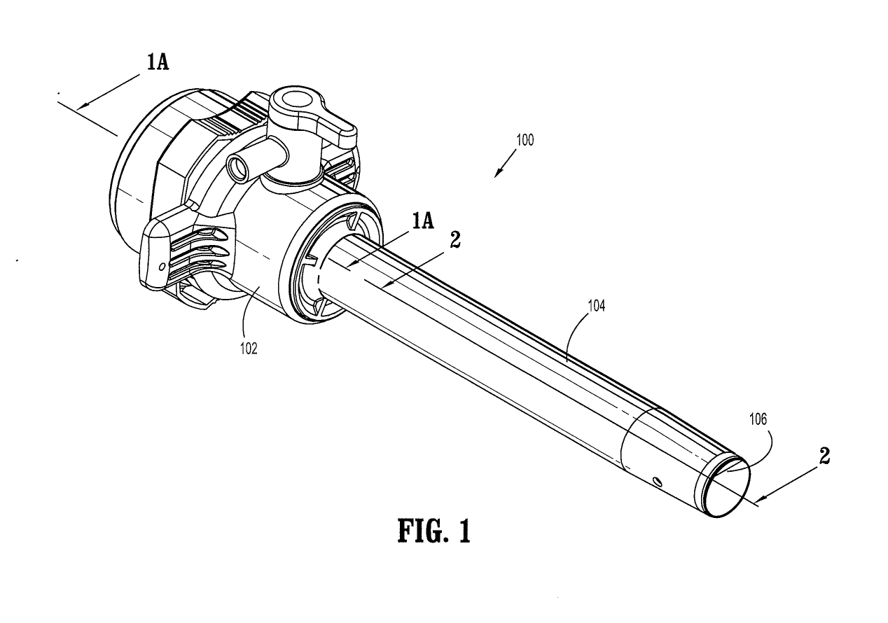

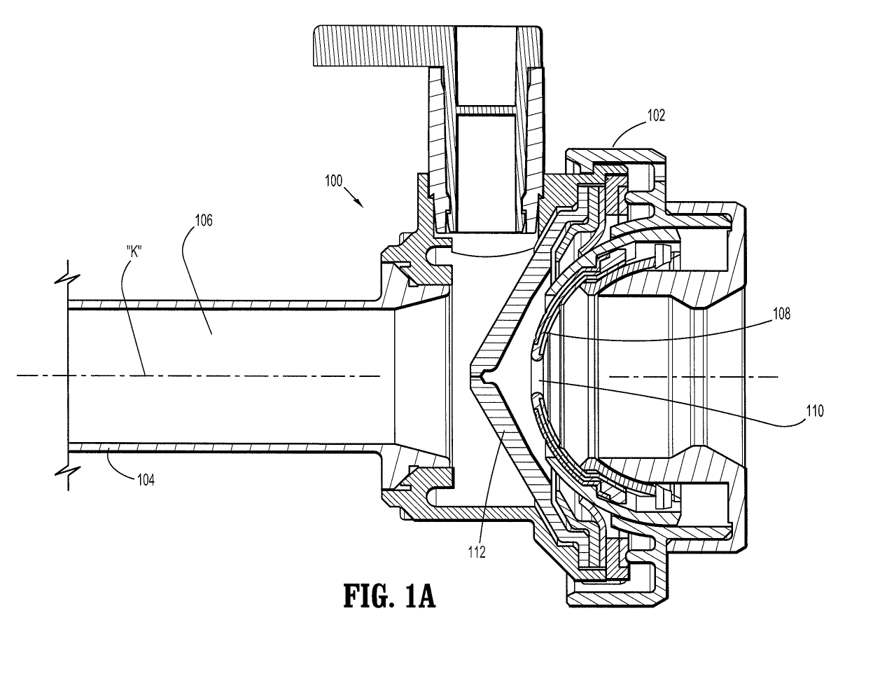

Referring now to the drawings, in which like reference numerals identify identical or substantially similar parts throughout the several views, FIGS. 1 and 1A illustrate the access apparatus 100 of the present disclosure. Access apparatus 100 may be any member suitable for the intended purpose of accessing a body cavity and typically defines a passageway permitting introduction of instruments or the clinician's hand therethrough. Access apparatus 100 is particularly adapted for use in laparoscopic surgery where the peritoneal cavity is insufflated with a suitable gas, e.g., CO₂, to raise the cavity wall from the internal organs therein. Access apparatus 100 is typically used with an obturator assembly (not shown) which may be blunt, a non-bladed, or a sharp pointed instrument positionable within the passageway of the access apparatus 100. The obturator assembly is utilized to penetrate the abdominal wall to introduce the access apparatus 100 through the abdominal wall, and then subsequently is removed from the access apparatus 100 to permit introduction of the surgical instrumentation utilized to perform the procedure through the passageway.

Access apparatus 100 includes housing member 102 and portal member 104 connected to the housing member 102 and extending therefrom. Portal member 104 defines a longitudinal axis “k” extending along the length of the portal member 104. Housing member 102 and portal member 104 further define internal longitudinal passage 106 dimensioned to permit passage of surgical instrumentation. Portal member 104 may be formed of any suitable medical grade material, such as stainless steel or other rigid materials, including polymeric materials, such as polycarbonate, or the like. Portal member 104 may be transparent or opaque. The diameter of portal member 104 may vary, but typically ranges from about 4.5 millimeters (mm) to about 15 millimeters (mm).

Housing member 102 may include a number of components assembled together to define the outer housing shown in the drawings. Housing member 102 includes object seal 108 which is adapted to establish a substantial seal about a surgical object, e.g., a surgical instrument. Object seal 108 may be a seal having an inner area or inner surfaces defining central aperture 110 for sealed reception of a surgical instrument. In one embodiment, object seal 108 is the gimbal seal disclosed in commonly assigned U.S. patent application Ser. No. 11/098966, filed Apr. 5, 2005, the entire contents of such disclosure being hereby incorporated by reference herein. Object seal 108 may comprise an elastomeric material and may, or may not, include a fabric layer juxtaposed with the elastomeric material. For example, in one embodiment, object seal 108 desirably comprises an elastomeric material compression-molded with a fabric material such as disclosed in certain embodiments of the commonly assigned U.S. Pat. No. 6,702,787 to Racenet, the entire contents of such disclosure being hereby incorporated by reference herein. The fabric may comprise a woven, knitted, braided, or non-woven material of polymeric materials. Alternatively, object seal 108 may comprise a gel material fabricated from soft urethane gel, silicon gel, etc.

Housing member 102 further includes valve 112. Valve 112 may be a zero-closure valve such as duck-bill valve having a slit which is adapted to close in the absence of a surgical object and/or in response to insufflation gases of the pressurized cavity. In the alternative, valve 112 may be a gel seal, balloon valve, or a flapper valve.

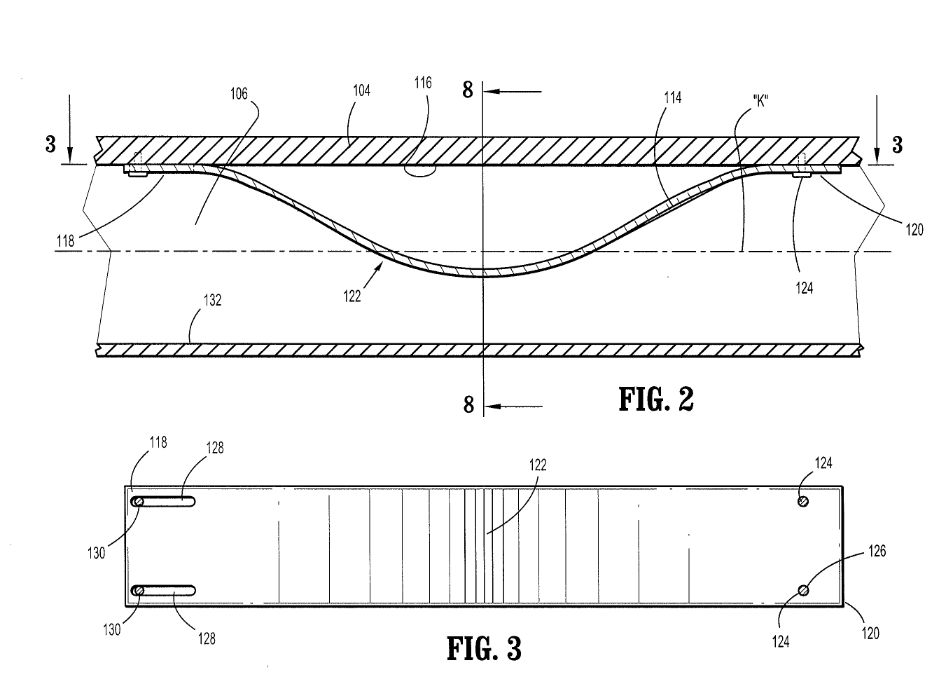

Referring now to FIGS. 2-4, one embodiment of the constraining mechanism of the present disclosure is illustrated. Constraining mechanism is adapted to constrain or restrict movement of the surgical object “o” within portal member 104. In embodiments, the constraining mechanism is adapted to bias the surgical object “o” in, e.g., a radial outward direction such that the surgical object “o” is restrained against structure within portal member 104. In this manner, the surgical object “o” may be secured in a fixed longitudinal position within portal member 104 thereby minimizing the potential for the surgical object “o” to be dislodged or expelled from access apparatus 100. In addition, the constraining mechanism may limit off-axis or angulated movement of the surgical object “o” thereby assisting in maintaining a seal with object seal 108 about the surgical object “o”. Even further, the constraining mechanism will also permit the clinician to leave the surgical object unattended during the performance of other surgical tasks. In the embodiments of FIGS. 2-4, the constraining mechanism includes elongated spring 114 mounted to an internal wall surface 116 of portal member 104. Elongated spring 114 may comprise any suitable resilient material such as stainless steel, titanium or a resilient polymeric material. Elongated spring 114 includes leading (distal) and trailing (proximal) ends 118, 120 respectively and an intermediate segment 122 disposed between the leading and the trailing ends 118, 120. Intermediate segment 122 includes a generally bowed segment having an arch or curvature extending toward the central longitudinal axis “k”.

Elongated spring 114 may be mounted to portal member 104 through a variety of mechanisms. In one embodiment, trailing or proximal end 120 is secured to internal wall surface 116 with at least one fastener or pin 124, which extends through a corresponding aperture 126 adjacent the trailing end. Two fasteners 124 and corresponding apertures 126 may be provided. Leading or distal end 118 is secured to internal wall surface 116 in a manner which permits the leading end 118 to move in a limited longitudinal direction with respect to longitudinal axis “k”. For example, elongated spring 114 includes a pair of elongated slots 128 adjacent leading end 118. Pins or fasteners 130 extend through slots 128 and are adapted to traverse the slots 128 during flexing of elongated spring 114.

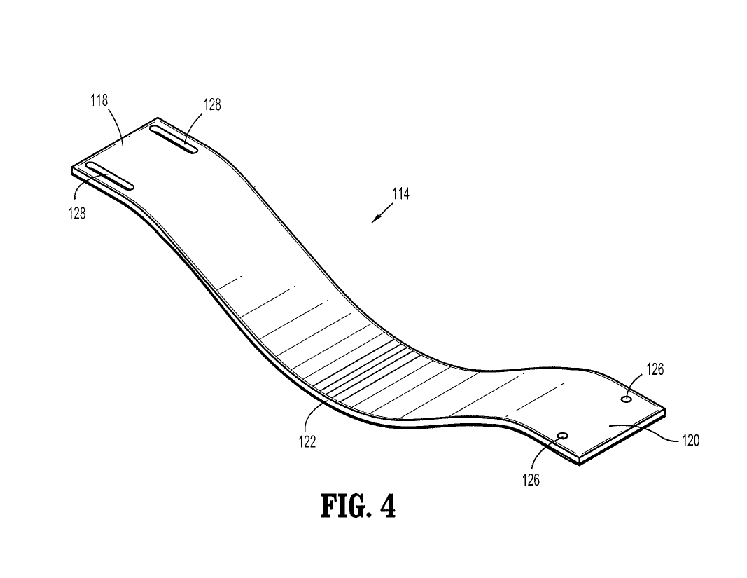

Elongated spring 114 is normally biased to an initial condition in which intermediate bowed segment 122 assumes the arrangement depicted in FIGS. 2 and 4. Intermediate bowed segment 122 may depend outwardly such that it engages the opposed wall surface 132 of portal member 104. In the alternative, bowed segment 122 may be displaced from opposed wall surface 132 when in the initial condition. Bowed segment 122 is dimensioned to intersect longitudinal passage 106 and the central longitudinal axis “k” to engage the surgical object.

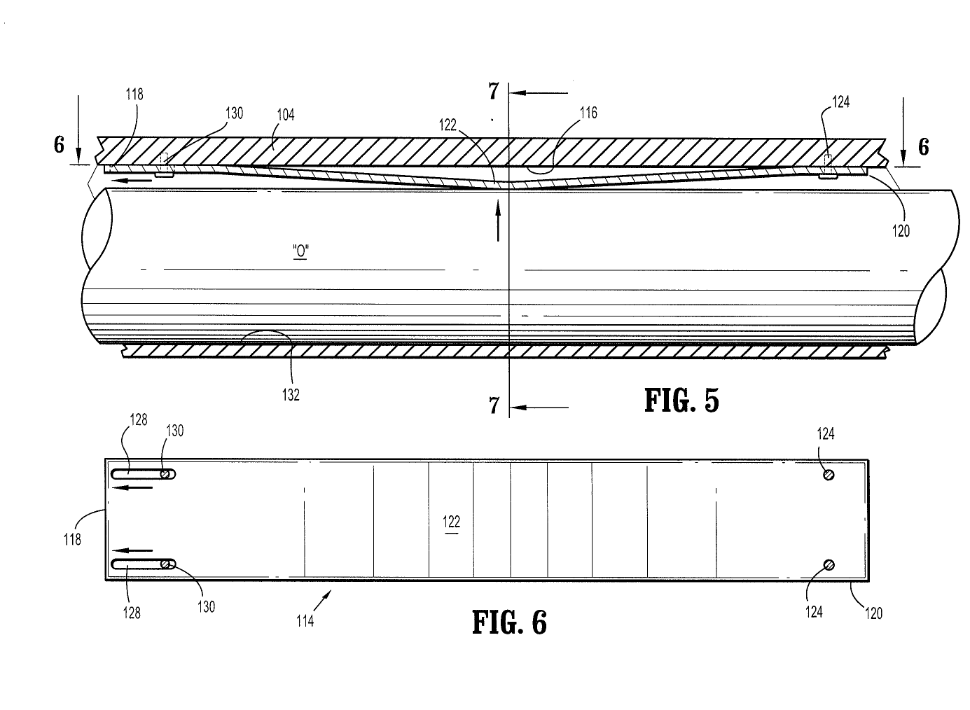

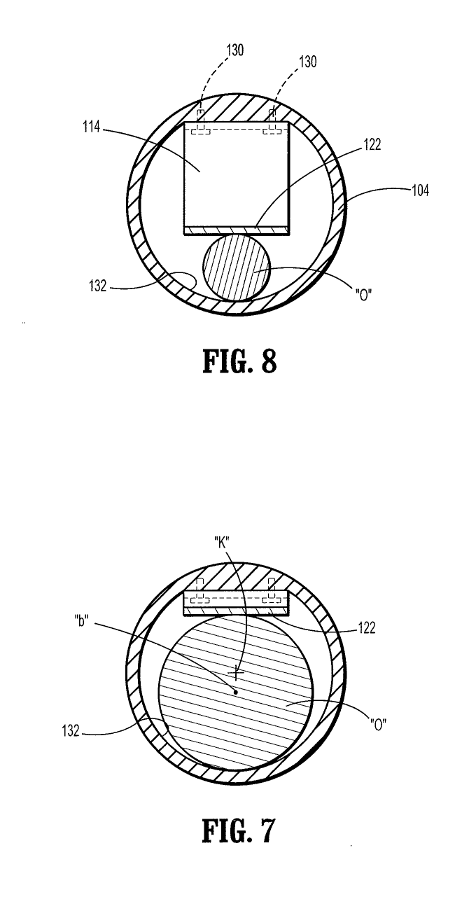

Referring now to FIGS. 5-7, the operation of the constraining mechanism will be discussed. During the course of a surgical procedure, a surgical object “o” will be introduced through access apparatus 100. As the surgical object “o” advances within portal member 104, bowed segment 122 contacts or engages the surgical object “o”. Further advancing movement of the surgical object “o” will cause bowed segment 122 to flex or deflect radially outwardly relative to central longitudinal axis “k” toward internal wall surface 116 and assume the stressed condition depicted in FIGS. 5 and 7. Concurrently therewith, leading end 118 will move in a distal or leading direction to accommodate the deflection of bowed segment 122 with pins or fasteners 130 traversing elongated slots 128 of elongated spring 114. As discussed, elongated spring 114 is normally biased to its initial condition. Accordingly, bowed segment 122 will continually urge the surgical object “o” in an opposed radial outward direction toward internal wall surface 132. In one embodiment, bowed segment 122 traps or constrains the surgical object “o” against the opposed internal wall surface 132 of portal member 104 to secure the surgical object “o” at a predefined longitudinal position. In this position, the axis “b” of the surgical object “o” may be in general parallel relation with longitudinal axis “k” of portal member 104 which may assist in maintaining a seal about the surgical object “o” by resisting angulation of the surgical object “o”. Upon removal of the surgical object “o”, bowed segment 122 will assume its initial condition of FIG. 2. FIG. 8 illustrates bowed segment 122 acting in concert with internal wall surface 132 of portal member 104 to secure a surgical object “o” of a relatively small diameter.

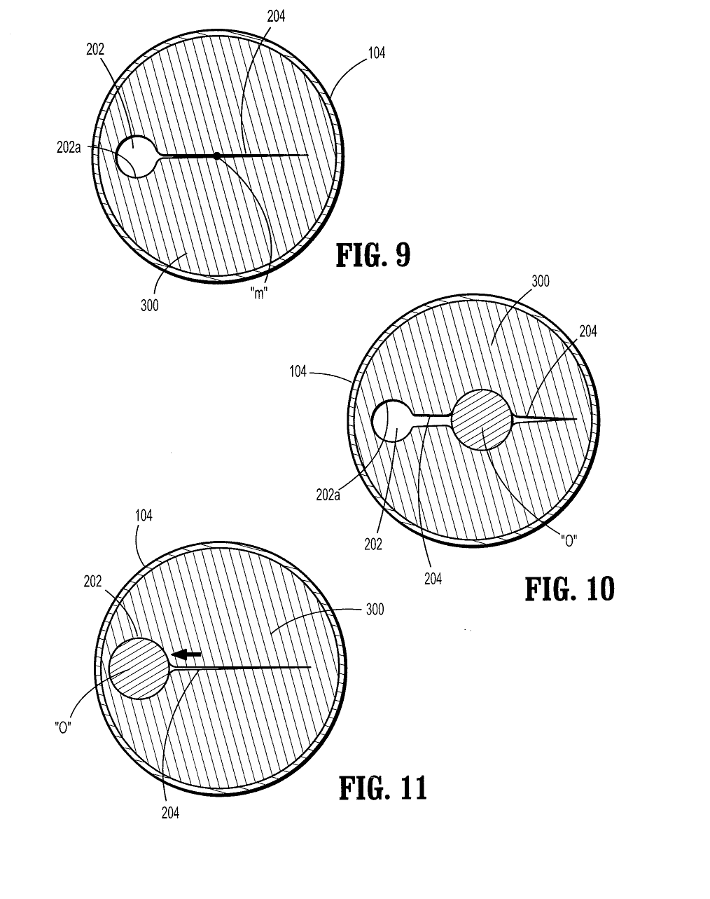

FIGS. 9-11 illustrate another alternate embodiment of the present disclosure. In accordance with this embodiment, constraining member 200 is generally disk shaped and may be mounted to portal member 104 by any conventional means including adhesives, cements or mechanical means. Constraining member 200 may be secured to portal member 104 at a longitudinal fixed position. Constraining member 200 may be fabricated from any of the suitable resilient materials identified hereinabove in connection with the discussion of elongated spring. Constraining member 200 includes opening 202 and internal surfaces 204 communicating with the opening 202. Opening 202 is radially displaced from the center “m” of constraining member 200 and with respect to the central longitudinal axis “k” of portal member 104. Internal surfaces 204 may taper away from each other as the internal surfaces 204 approaches opening 202. FIG. 9 illustrates the initial condition of constraining member 200. In this position, internal surfaces 204 are in an approximated relationship.

In use, surgical object “o” is introduced within the access apparatus 100 and advanced within portal member 104 to engage constraining member 200. Internal surfaces 204 of constraining member 200 will deflect outwardly relative to accommodate passage of the surgical object “o” to assume the stressed condition depicted in FIG. 10. However, because internal surfaces 204 are normally biased to the initial condition of FIG. 9, the internal surfaces 204 act in concert to apply a radial force against the outer circumference of the surgical object “o”. The application of the radial force about the circumference of the surgical object “o” will drive the surgical object “o” towards aperture 202, and may be facilitated through the tapered arrangement (if present) of internal surfaces 204. The surgical object “o” thereby is received within aperture 202 with the inner surfaces 202a defining the aperture circumscribing the surgical object “o”. The surgical object “o” is constrained within the aperture 202. In one embodiment, the surgical object “o” may be secured at a longitudinal fixed position within aperture 202 through frictional engagement with inner surfaces 202a defining aperture 202. in the restrained condition, the axis “b” of the surgical object “o” may be in parallel relation with the longitudinal axis “k” of portal member 104, thereby assisting in maintaining the seal about the surgical object “o”.

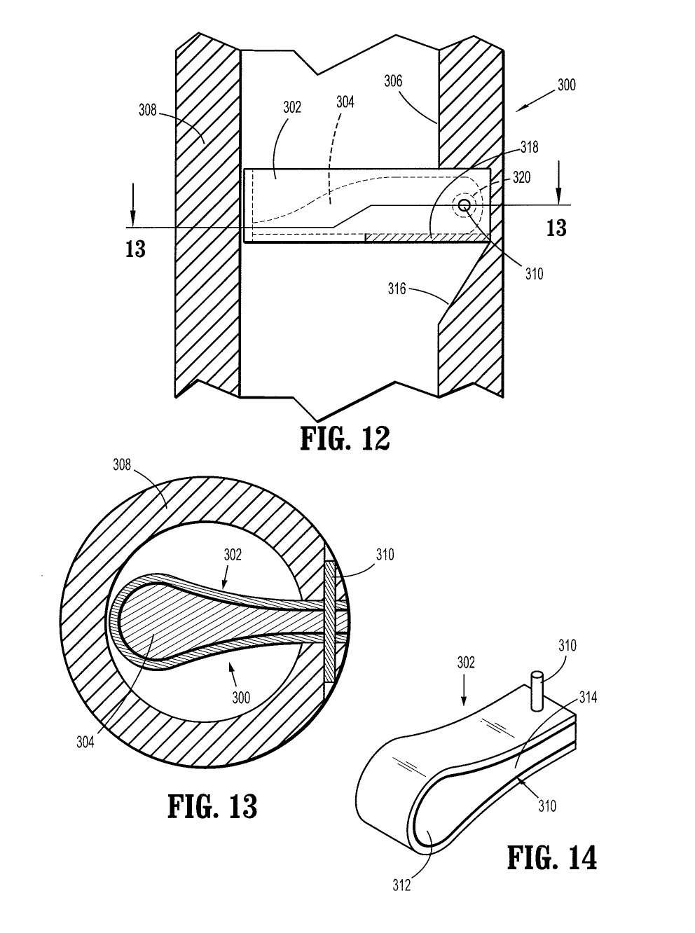

FIGS. 12-14 illustrate another embodiment of the constraining mechanism 300 in accordance with the present disclosure. The constraining mechanism 300 includes eyelet 302 and lever 304 both of which are mounted within an internal surface 306 of portal member 308. In one embodiment, both eyelet 302 and lever 304 define apertures for reception of mounting pin 308 which is secured to, or embedded within, portal member 308. As best depicted in FIG. 14, eyelet 302 defines an elongated or oblong opening 310 having a circular segment 312 displaced from mounting pin 308 and a tapered segment 314 adjacent the mounting pin 300.

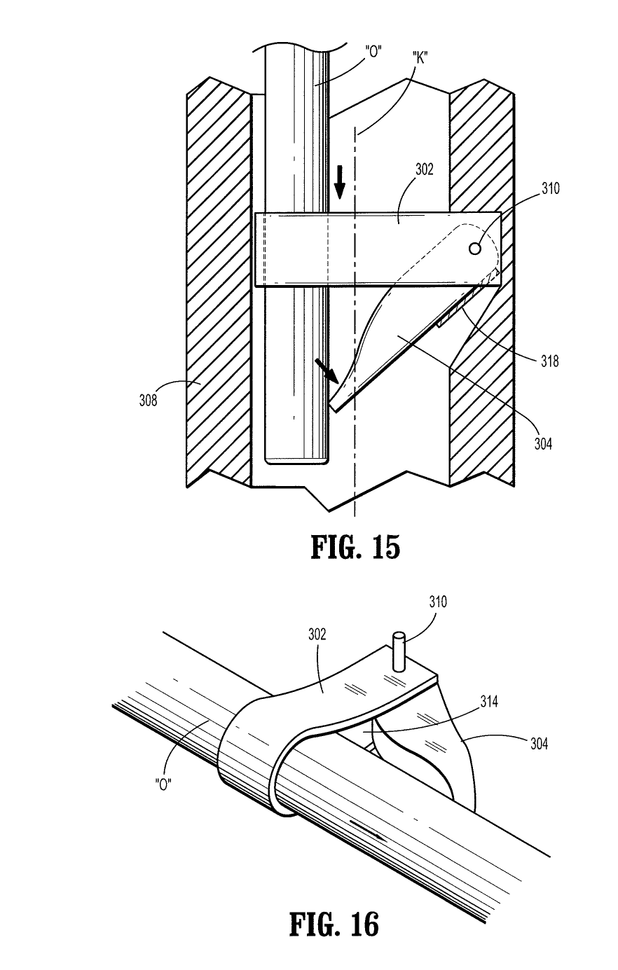

Lever 304 is adapted for pivotal movement about mounting pin 308 and relative to eyelet 302 between the initial condition depicted in FIGS. 12-13 and the pivoted position depicted in FIGS. 15 and 16. In one embodiment, portal member 308 includes recessed section 316 to permit unobstructed pivoting movement of lever 304. Lever 304 is normally biased toward the initial condition through a biasing mechanism. For example, in one embodiment, a leaf spring 318 is mounted to portal member 308 and engages the underside of lever 304. Leaf spring 318 will bias lever 304 in a vertical upward or counterclockwise direction (FIG. 12) i.e., toward the initial condition. In another embodiment, a torsion spring 320 (shown in phantom) may be coaxially arranged about mounting pin 310. Torsion spring 320 may include one end mounted or embedded within portal member 104 and another end engageable with the underside of lever 304. In a further embodiment, lever 304 is monolithically formed with portal member 104 and pivots about a living hinge between the initial and stressed conditions. Lever 304 may be fabricated from a suitable metal or polymeric material.

In use, the surgical object “o” is introduced through eyelet 302 and engages lever 304. Lever 304 is deflected to pivot about mounting pin 308 to assume the stressed condition and permit passage of the surgical object “o” as depicted in FIG. 15. The normal bias of lever 304 toward the initial condition will cause the lever 304 to engage and drive the surgical object “o” radially outwardly relative to the central axis “k” of portal member 104 and position the surgical object “o” within the circular segment 312 of eyelet 302. Circular segment 302 may be dimensioned to at least partially circumscribe the surgical object “o” to further restrain the surgical object “o” within the circular segment 302 of eyelet 302. Circular segment 302 may also be dimensioned to frictionally engage the surgical object “o” in a manner to secure, either individually, or in concert with lever 304, the surgical object “o” at a fixed position within portal member 308 and relative to the longitudinal axis “k”, and possibly parallel with the longitudinal axis “k”. Once the surgical procedure or task is complete, the surgical object “o” may be withdrawn from portal member to permit lever 304 to assume the initial condition of FIG. 12. In the initial condition, eyelet 302 and lever 304 may function as a closure valve preventing the escape of gases form the underlying body cavity

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

What is claimed is:

1. A surgical access apparatus, which comprises:
a housing member;
a portal member extending from the housing member and defining a central longitudinal axis, the housing member and the portal member defining a longitudinal passage therethrough dimensioned to permit passage of an surgical object; and
a constraining mechanism associated with at least one of the housing member and the portal member, the constraining mechanism including a constraining member dimensioned and positioned to intersect the longitudinal passage, the constraining member adapted to engage the surgical object and bias the surgical object toward a position radially displaced relative to the central longitudinal axis.

2. The surgical access apparatus according to claim 1 wherein the constraining member is adapted to transition from an initial condition in the absence of the surgical object to a stressed condition in the presence of the surgical object, the constraining member normally biased toward the initial condition.

3. The surgical access apparatus according to claim 2 wherein the constraining member includes an elongated spring member.

4. The surgical access apparatus according to claim 3 wherein the elongated spring member is at least partially disposed within the portal member.

5. The surgical access apparatus according to claim 4 wherein the elongated spring member includes leading and trailing ends, one of the leading and trailing ends mounted in fixed relation to the portal member, the other of the leading and trailing end mounted for longitudinal movement relative to the portal member.

6. The surgical access apparatus according to claim 5 wherein the elongated spring member defines a bowed segment between the leading and trailing ends.

7. The surgical access apparatus according to claim 4 wherein the elongated spring member is dimensioned to engage and substantially restrain the surgical object against an inner wall surface of the portal member.

8. The surgical access apparatus according to claim 2 wherein the constraining member defines a substantially disc shaped member, the disc shaped member defining an opening therethrough for substantially restraining the surgical object, the opening radially displaced with respect to the central longitudinal axis of the portal member, the disc shaped member further having inner surfaces defining an object passage in communication with the opening, the inner surfaces dimensioned and configured to bias the surgical object toward the opening.

9. The surgical access apparatus according to claim 8 wherein the inner surfaces are arranged in to taper inwardly toward the opening.

10. The surgical access apparatus according to claim 2 wherein the constraining member includes a lever mounted within one of the housing and the portal member, the lever mounted for pivotal movement between a first position corresponding to the initial condition of the constraining member and a second position corresponding to the stressed condition of the constraining member.

11. The surgical access apparatus according to claim 10 wherein the lever is normally biased toward the first position.

12. The surgical access apparatus according to claim 11 including an eyelet defining an opening for passage of the surgical object, the lever dimensioned to engage and substantially restrain the surgical object against an inner wall surface of the eyelet.

13. The surgical access apparatus according to claim 12 wherein the lever is dimensioned to engage the eyelet, the lever and the eyelet being dimensioned to substantially close the longitudinal passage of the portal member.

14. The surgical access apparatus according to 1 including an object seal within the housing, the object seal dimensioned to establish a substantial seal about the surgical object.

15. The surgical access apparatus according to 1 including a zero closure valve adapted to open to permit passage of the surgical object and to close in the absence of the surgical object.
